(19) United States
(12) Patent Application Publication
(10) Pub. No.: US 2020/0030463 A1
(43) Pub. Date: Jan. 30, 2020

(54) USE RADIUM RA-223 DICHLORIDE FOR THE TREATMENT OF MULTIPLE MYELOMA

(71) Applicants: Bayer Aktiengesellschaft, Leverkusen (DE); Bayer Pharma Aktiengesellschaft, Berlin (DE)

(72) Inventor: Arne SCHOLZ, Berlin (DE)

(21) Appl. No.: 16/484,312

(22) PCT Filed: Feb. 20, 2018
(86) PCT No.: PCT/EP2018/054110

§ 371 Date: Aug. 7, 2019

(30) Foreign Application Priority Data
Feb. 24, 2017 (EP) 17157869.3

(51) Int. Cl. A61K 51/00; A61P 35/00; A61K 31/69

(52) U.S. Cl. CPC A61K 51/00; A61K 31/69; A61P 35/00

(57) ABSTRACT

The present invention relates to the use of radium-223, particularly a pharmaceutically acceptable salt of radium-223, and combinations comprising radium-223, for the preparation of a medicament for the treatment or prophylaxis of a haematologic malignancy disease, particularly for the treatment of multiple myeloma thereof.

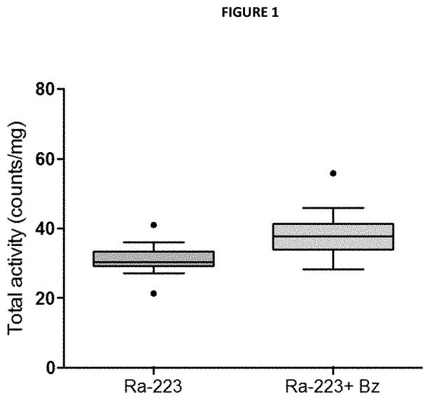

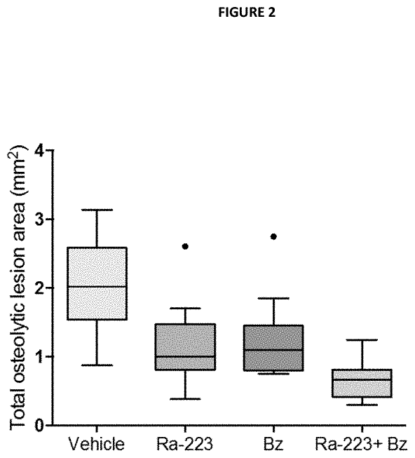

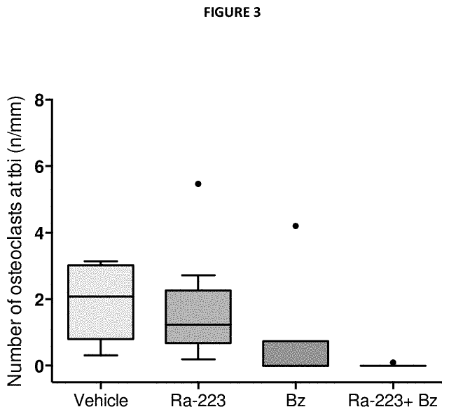

The present invention relates to the use of radium-223, particularly a pharmaceutically acceptable salt of radium-223, and combinations comprising radium-223, for the preparation of a medicament for the treatment or prophylaxis of a haematologic malignancy disease, particularly for the treatment of multiple myeloma thereof.

### BACKGROUND OF THE INVENTION

Xofigo® uses alpha radiation from radium-223 decay to kill cancer cells. Xofigo targets to bone tissue by virtue of its chemical similarity to calcium. It has an effect over a range of 2-10 cells and causes less damage to surrounding healthy tissues compared to current radiation therapy based on beta or gamma radiation. Significant increase in median overall survival was demonstrated in Phase III clinical trials and Xofigo was approved as a treatment for castration-resistant prostate cancer (CRPC) patients with symptomatic bone metastases.

A preferred suitable pharmaceutically acceptable salt of radium-223 is the dichloride (Ra²²³Cl₂). Radium-223 dichloride is a novel, targeted alpha-emitter that selectively binds to areas of increased bone turnover in bone metastases and emits high-energy alpha-particles of extremely short (<100 μm) range (Bruland O. S. et al., High-linear energy transfer irradiation targeted to skeletal metastases by the alpha-emitter ²²³Ra: adjuvant or alternative to conventional modalities?, Clin. Cancer Res. 2006; 12: 6250s-7s). It is the first targeted alpha-emitter approved for the treatment of prostate cancer with bone metastases.

As a bone-seeking calcium mimetic, radium-223 is bound into newly formed bone stroma, especially within the microenvironment of osteoblastic or sclerotic metastases. (Henriksen G. et al., Significant antitumor effect from bone-seeking, alpha-particle-emitting (223)Ra demonstrated in an experimental skeletal metastases model, Cancer Res. 2002; 62: 3120-3125; Henriksen G. et al., Targeting of osseous sites with alpha-emitting 223Ra: comparison with the beta-emitter 89Sr in mice, J. Nucl. Med 2003; 44: 252-59). The high-energy alpha-particle radiation induces mainly double-strand DNA breaks resulting in a potent and highly localized cytotoxic effect in the target areas containing metastatic cancer cells (Lewington V. J., Bone-seeking radionuclides for therapy, J. Nucl. Med 2005; 46 (suppl 1): 38S-47S; Liepe K., Alpharadin, a ²²³Ra-based alpha-particle-emitting pharmaceutical for the treatment of bone metastases in patients with cancer, Curr. Opin. Investig. Drugs 2009; 10: 1346-58; McDevitt M. R. et al., Radioimmunotherapy with alpha-emitting nuclides, Eur. J. Nucl. Med. 1998; 25: 1341-51). The short path length of the alpha-particles also means that toxicity to adjacent healthy tissue and particularly the bone marrow may be reduced (Kerr C., (223)Ra targets skeletal metastases and spares normal tissue, Lancet Oncol. 2002; 3: 453; Li Y., Russell P. J., Allen B. J., Targeted alpha-therapy for control of micrometastatic prostate cancer, Expert Rev. Anticancer Ther. 2004; 4: 459-68).

Radium-223 has demonstrated a favorable safety profile with minimal myelotoxicity in phase 1 and 2 studies of patients with bone metastases (Nilsson S. et al., First clinical experience with alpha-emitting radium-223 in the treatment of skeletal metastases, Clin. Cancer Res. 2005; 11: 4451-59; Nilsson S. et al., Bone-targeted radium-223 in symptomatic, hormone-refractory prostate cancer: a randomised, multicentre, placebo-controlled phase II study, Lancet Oncol. 2007; 8: 587-94).

Phase 2 studies have shown that radium-223 reduces pain, improves disease-related biomarkers (e.g., bone alkaline phosphatase [ALP] and prostate-specific antigen [PSA]), and have suggested a survival benefit in patients with CRPC and bone metastases (Parker C. et al., A randomized, double-blind, dose-finding, multicenter, phase 2 study of radium chloride (Ra-223) in patients with bone metastases and castration-resistant prostate cancer, Eur Urol. 2013 February; 63(2):189-97; Nilsson S. et al., A randomized, dose-response, multicenter phase II study of radium-223 chloride for the palliation of painful bone metastases in patients with castration-resistant prostate cancer, Eur. J. Cancer 2012; 48: 678-86).

The ALSYMPCA (ALpharadin in SYMptomatic Prostate CAncer patients) trial provides proof of principle for the role of targeted alpha-emitters in oncology. In this trial, radium-223 significantly prolonged overall survival with a 30.5% reduction in risk of death compared with placebo in patients with CRPC (Castration Resistant Prostate Cancer) and bone metastases. Median survival with radium-223 was longer than placebo by 2.8 months. All main secondary efficacy endpoints were statistically significant and favored treatment with radium-223, including the clinically defined endpoint of time to first skeletal-related event, which was significantly prolonged in patients receiving radium-223 (C. Parker et al., Alpha Emitter Radium-223 and Survival in Metastatic Prostate Cancer, The New England Journal of Medicine 369(3):213-23).

²²³Ra is used as an α-emitting radiopharmaceutical for targeting of calcified tissues, e.g. bone surfaces and osseous tumor lesions. It can be suitable as a bone seeking radiopharmaceutical. It thus may be used for prophylactic cancer treatment by delivering a focused dose to bone surfaces in patients with a high probability of having undetected micrometastases at bone surfaces. Another example of its potential use would be in the treatment of painful osseous sites. The alkaline-earth radionuclide radium-223 is useful for the targeting of calcified tissues, e.g., bone and a pharmaceutical acceptable solution comprising ²²³Ra. The alkaline-earth radionuclide radium-223 is suitable for the use of the nuclide as a cationic species and/or associated to a chelator or another form of a carrier molecule with affinity for calcified tissues. Thus may be combined with a chelator that can be subsequently conjugated to a molecule with affinity for calcified tissues. The effect of the radioisotope generated by providing a cascade of α-particles on bone surfaces and/or in calcified tumors for the palliation of pain caused by various diseases and/or for the prophylactic use against possible minimal disease to the skeleton, and/or also for the therapeutic treatment of established cancer to the bone.

Multiple myeloma is the second most common haematologic malignancy, with 20,000 new cases per year (Jemal A, et al, Cancer J. Clin., 2007, 57: 43-66), and remains incurable with a median survival of 3 to 5 years (Kyle R A, Rajkumar S V. Multiple myeloma. N. Engl. J. Med., 2004, 351: 1860-73).

Further, as multiple myeloma is a plasma cell malignancy characterised by complex heterogeneous cytogenetic abnormalities, the bone marrow microenvironment promotes multiple myeloma cell growth and resistance to conventional therapies.

The present invention is thus to provide compounds for the preparation of a medicament for use in the treatment or prophylaxis of multiple myeloma.

The state of the art does not disclose the use of a pharmaceutically acceptable salt of the alkaline-earth radionuclide radium-223 to for the treatment of multiple myeloma.

### DETAILED DESCRIPTION OF THE INVENTION

A first aspect of the present invention relates to the use of radium-223 dichloride as a sole active agent, or of a pharmaceutical composition containing radium-223 dichloride for the preparation of a medicament for the treatment or prophylaxis of multiple myeloma.

A second aspect of the present invention relates to a combination of:

a) a) (1R)-3-methyl-1-[[(2S)-1-oxo-3-phenyl-2-[(pyrazinylcarbonyl)amino]propyl]-amino]butyl]boronic acid (hereinafter, including the claims: “bortezomib”) or a pharmaceutically acceptable salt or hydrate thereof,

and

b) radium-223 dichloride as a sole active agent, or of a pharmaceutical composition containing radium-223 dichloride.

In accordance with another aspect, the present invention covers a combination of any component A mentioned herein with any component B mentioned herein, optionally with any component C mentioned herein.

In accordance with a further aspect, the present invention concerns combinations of at least two components A and B, preferably of two components, component A being bortezomib or a pharmaceutically acceptable salt or hydrate thereof, and component B being pharmaceutically acceptable salt of the alkaline-earth radionuclide radium-223.

In accordance with another aspect, the present invention provides combinations of at least two components A and B, preferably of two components, component A being bortezomib or a pharmaceutically acceptable salt or hydrate thereof or a pharmaceutically acceptable salt thereof, and component B being a pharmaceutically acceptable inorganic salt of the alkaline-earth radionuclide radium-223.

The combinations comprising at least two components A and B, preferably two components, as described and defined herein, are also referred to as “combinations of the present invention”.

The synergistic behavior of a combination of the present invention is demonstrated herein with bortezomib or a pharmaceutically acceptable salt or hydrate thereof (“Compound A”) specifically disclosed in the Examples section.

Bortezomib can inhibit proteosome in organisms. Bortezomib is believed to function as a reversible inhibitor of the chymotrypsin-like activity of the 26S proteasome in mammalian cells. The 26S proteasome is a large protein complex that degrades ubiquitinated proteins. The ubiquitin-proteasome pathway plays a role in regulating the intracellular concentration of specific proteins, maintaining homeostasis within cells. Inhibition of the 26S proteasome prevents this targeted proteolysis, which can affect multiple signaling cascades within the cell. This disruption of normal homeostatic mechanisms can lead to cell death.

Bortezomib is cytotoxic to a variety of cancer cell types in vitro and causes a delay in tumor growth in vivo in nonclinical tumor models, including multiple myeloma. Bortezomib presently is approved for the treatment of multiple myeloma, relapsed multiple myeloma, and mantle cell lymphoma. A variety of combination therapies have been investigated for treating multiple myeloma, in which bortezomib is administered with one or more other biologically active substances, such as lenalidomide, dexamethasone, melphalan, predisone, thalidomide, cyclophosphamide, doxorubicin, vincristine, carmustine, pomalidomide, vorinostat, tanespimycin, and perifosine. Other potential uses of bortezomib also have been reported, including treatment of amyloidosis.

A disadvantage that bortezomib shares with other peptidyl boronic acids and esters is an instability to standard conditions of purification and storage. Boronic acids and esters tend to form anhydrides, including cyclic anhydrides referred to as “boroxines,” during dehydration, which can make it difficult to purify the desired compound. Boronic acids and esters also tend to oxidize in air, which can severely limit their shelf life. Thus, bortezomib typically is difficult to purify, to characterize and/or to formulate into a stable therapeutic product.

One conventional method of increasing the stability of bortezomib involves combining the boronic acid with a sugar or other compound having two or more hydroxyl groups separated by at least two connecting atoms (i.e. C, N, S or O). See, for example, U.S. Pat. No. 6,699,835 to Plamondon et al. It is reported that bortezomib forms a boronate ester with such a di-hydroxyl compound, and that this ester is more stable to air and to dehydration than bortezomib alone. Preferred di-hydroxyl compounds for this stabilization method are disclosed as the reduced sugars sorbitol and mannitol. In a specific embodiment of this method, a mixture of bortezomib, the sugar and a solvent is subjected to lyophilization to remove the solvent, providing a powder containing the bortezomib, the sugar and/or an ester of the bortezomib and the sugar.

This sugar stabilization method has been implemented in the formulation that is commercially available at present and is sold under the VELCADE® trademark. VELCADE®. for Injection (Millennium Pharmaceuticals, Inc.; Cambridge, Mass., USA) is currently available as a lyophilized powder containing bortezomib and mannitol. A single dose of VELCADE® includes 3.5 milligrams (mg) bortezomib and 35 mg mannitol. VELCADE® is reconstituted by combining the lyophilized powder with 3.5 milliliters (mL) of 0.9% sodium chloride saline, to provide an injectable solution having a bortezomib concentration of 1 mg/mL.

In addition, a combination of the present invention comprising Compound A as mentioned above and a pharmaceutically acceptable salt of radium-223, particularly 223RaCl₂, is a preferred aspect of the invention.

In another aspect a combination of the present invention comprises Compound A or a pharmaceutically acceptable salt thereof and a pharmaceutically acceptable salt of the alkaline-earth radionuclide radium-223, preferably the dichloride salt of radium-223.

In a preferred embodiment the combination of the present invention comprises Compound A or a pharmaceutically acceptable salt thereof and the dichloride salt of radium-223.

Further, the present invention covers a kit comprising:

component A: bortezomib or a pharmaceutically acceptable salt or hydrate thereof, or a stereoisomer, a tautomer, an N-oxide, a hydrate, a solvate, or a pharmaceutically acceptable salt thereof;

component B: a pharmaceutically acceptable salt of the alkaline-earth radionuclide radium-223 or a solvate or a hydrate thereof as described supra.

In the kit optionally either or both of said components A and B in any of the above-mentioned combinations are in the form of a pharmaceutical composition which is ready for use to be administered simultaneously, concurrently, separately or sequentially. The components A and B may be administered independently of one another by the oral, intravenous, topical, local installations, intraperitoneal or nasal route. Preferably component A is administered by the oral route and component B is administered by the intravenous route.

Further, the present invention covers a kit comprising:

component A: bortezomib or a pharmaceutically acceptable salt or hydrate thereof, or a stereoisomer, a tautomer, an N-oxide, a hydrate, a solvate, or a pharmaceutically acceptable salt thereof;

component B: a pharmaceutically acceptable salt of the alkaline-earth radionuclide radium-223 or a solvate or a hydrate thereof as described supra; and, optionally,

component C: one or more, preferably one, further pharmaceutical agent(s), in which optionally either or all of said components A, B and C in any of the above-mentioned combinations are in the form of a pharmaceutical composition which is ready for use to be administered simultaneously, concurrently, separately or sequentially. The components A and B, optionally C, may be administered independently of one another by the oral, intravenous, topical, local installations, intraperitoneal or nasal route.

The term “component C” being at least one pharmaceutical agent includes the effective compound itself as well as its pharmaceutically acceptable salts, solvates, hydrates or stereoisomers as well as any pharmaceutical composition comprising such effective compound or its pharmaceutically acceptable salts, solvates, hydrates or stereoisomers. A list of such pharmaceutical agents of component C is being provided further below.

The combinations of component A and component B of this invention can be administered as the sole pharmaceutical agent or in combination with one or more further pharmaceutical agents C where the resulting combination of components A, B and C causes no unacceptable adverse effects. For example, the combinations of components A and B of this invention can be combined with component C, i.e. one or more further pharmaceutical agents, such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

The compounds of this invention can be administered as the sole pharmaceutical agent or in combination with one or more other pharmaceutical agents (or “further active agents”) where the combination causes no unacceptable adverse effects. For example, the compounds of this invention can be combined with known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

The additional pharmaceutical agent or agents (or “further active agent”) can be, but are not limited to 131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin, or a combination thereof.

The additional pharmaceutical agent or agents (or “further active agent”) can be, but are not limited to aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubidine, chlorambucil, cisplatin, cladribine, cladribine, clodronic acid, cyclophosphamide, cytarabine, dacarbazine, dactinomycin, DaunoXome, decadron, decadron phosphate, delestrogen, denileukin diftitox, depo-medrol, deslorelin, dexomethasone, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine phosphate sodium, ethinyl estradiol, ethyol, etidronic acid, etopophos, etoposide, fadrozole, farston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron HCl, herceptin, histrelin, hycamtin, hydrocortone, eyrthro-hydroxynonyladenine, hydroxyurea, ibritumomab tiuxetan, idarubicin, ifosfamide, interferon alpha, interferon-alpha 2, interferon alfa-2A, interferon alfa-2B, interferon alfa-n1, interferon alfa-n3, interferon beta, interferon gamma-1a, interleukin-2, intron A, iressa, irinotecan, kytril, lapatinib, lentinan sulphate, letrozole, leucovorin, leuprolide, leuprolide acetate, lenalidomide, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, Mesna, methotrexate, metvix, miltefosine, minocycline, mitomycin C, mitotane, mitoxantrone, Modrenal, Myocet, nedaplatin, neulasta, neumega, neupogen, nilutamide, nolvadex, NSC-631570, OCT-43, octreotide, ondansetron HCl, orapred, oxaliplatin, paclitaxel, pediapred, pegaspargase, Pegasys, pentostatin, picibanil, pilocarpine HCl, pirarubicin, plicamycin, porfimer sodium, prednimustine, prednisolone, prednisone, premarin, procarbazine, procrit, refametinib (BAY 86-9766 (RDEA 119)), raltitrexed, rebif, rhenium-186 etidronate, rituximab, roferon-A, romurtide, salagen, sandostatin, sargramostim, semustine, sizofiran, sobuzoxane, solu-medrol, sparfosic acid, stem-cell therapy, streptozocin, strontium-89 chloride, sunitinib, synthroid, tamoxifen, tamsulosin, tasonermin, tastolactone, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran, ABI-007, acolbifene, actimmune, affinitak, aminopterin, arzoxifene, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifene, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thalidomide, thymosin alpha 1, tiazofurine, tipifarnib, tirapazamine, TLK-286, toremifene, TransMID-107R, valspodar, vapreotide, vatalanib, verteporfin, vinflunine, Z-100, zoledronic acid or combinations thereof.

In accordance with an embodiment, the additional pharmaceutical agent or agents (or “further active agent”) is selected from the group consisting of: 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 1000394, refametinib (BAY 86-9766 (RDEA 119)), belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, raloxifene, raltitrexed, ranimustine, razoxane, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

The additional pharmaceutical agent can also be gemcitabine, paclitaxel, cisplatin, carboplatin, sodium butyrate, 5-FU, doxirubicin, tamoxifen, etoposide, trastumazab, gefitinib, intron A, rapamycin, 17-AAG, U0126, insulin, an insulin derivative, a PPAR ligand, a sulfonylurea drug, an α-glucosidase inhibitor, a biguanide, a PTP-1B inhibitor, a DPP-IV inhibitor, a 11-beta-HSD inhibitor, GLP-1, a GLP-1 derivative, GIP, a GIP derivative, PACAP, a PACAP derivative, secretin or a secretin derivative.

Optional anti-hyper-proliferative agents which can be added to the composition include but are not limited to compounds listed on the cancer chemotherapy drug regimens in the 11th Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

Preferred optional pharmaceutical agents which may be added as component C to the combination of components A and B is/are one or more agents selected from enzalutamide, bicalutamide, flutamide, nilutamide, and/or abiraterone.

Generally, the use of cytotoxic and/or cytostatic agents as component C in combination with a combination of components A and B of the present invention may serve to:

(1) yield better efficacy in reducing the growth of a tumor and/or metastasis or even eliminate the tumor and/or metastasis as compared to administration of either agent alone,

(2) provide for the administration of lesser amounts of the administered chemo-therapeutic agents,

(3) provide for a chemotherapeutic treatment that is well tolerated in the patient with fewer deleterious pharmacological complications than observed with single agent chemotherapies and certain other combined therapies,

(4) provide for treating a broader spectrum of different cancer types in mammals, especially humans,

(5) provide for a higher response rate among treated patients,

(6) provide for a longer survival time among treated patients compared to standard chemotherapy treatments,

(7) provide a longer time for tumor progression, and/or

(8) yield efficacy and tolerability results at least as good as those of the agents used alone, compared to known instances where other cancer agent combinations produce antagonistic effects.

Further, the present invention covers a pharmaceutical composition comprising a combination of the present invention as described supra together with one or more pharmaceutically acceptable excipients.

Further, the present invention covers a pharmaceutical composition comprising a combination of at least two components, component A and component B, component A being bortezomib or a stereoisomer, a tautomer, an N-oxide, a hydrate, a solvate, or a pharmaceutically acceptable salt thereof, and component B being a pharmaceutically acceptable salt of the alkaline-earth radionuclide radium-223 together with one or more pharmaceutically acceptable excipients.

Further, the present invention covers a pharmaceutical composition comprising a combination of at least two components, component A and component B, component A being bortezomib or a stereoisomer, a tautomer, an N-oxide, a hydrate, a solvate, or a pharmaceutically acceptable salt thereof, and component B being a pharmaceutically acceptable salt of the alkaline-earth radionuclide radium-223, optionally with any component C mentioned herein, together with one or more pharmaceutically acceptable excipients.

In another embodiment the components A and B, and optionally component C, are present in separate formulations.

In another embodiment the components A and B, and optionally component C, are present in a joint formulation.

Pharmaceutically acceptable excipients are non-toxic, preferably they are non-toxic and inert. Pharmaceutically acceptable excipients include, inter alia,
fillers and excipients (for example cellulose, microcrystalline cellulose, such as, for example, Avicel®, lactose, mannitol, starch, calcium phosphate such as, for example, Di-Cafos®),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat)
solvents (for example water, ethanol, Isopropanol, glycerol, propylene glycol, medium chain-length triglycerides fatty oils, liquid polyethylene glycols, paraffins),
surfactants, emulsifiers, dispersants or wetters (for example sodium dodecyle sulphate, lecithin, phospholipids, fatty alcohols such as, for example, Lanette®, sorbitan fatty acid esters such as, for example, Span®, polyoxyethylene sorbitan fatty acid esters such as, for example, Tween®, polyoxyethylene fatty acid glycerides such as, for example, Cremophor®, polyoxethylene fatty acid esters, polyoxyethylene fatty alcohol ethers, glycerol fatty acid esters, poloxamers such as, for example, Pluronic®),
buffers and also acids and bases (for example phosphates, carbonates, citric acid, acetic acid, hydrochloric acid, sodium hydroxide solution, ammonium carbonate, trometamol, triethanolamine)
isotonicity agents (for example glucose, sodium chloride),
adsorbents (for example highly-disperse silicas)
viscosity-increasing agents, gel formers, thickeners and/or binders (for example polyvinylpyrrolidon, methylcellulose, hydroxypropylmethylcellulose, hydroxypropylcellulose, carboxymethylcellulose-sodium, starch, carbomers, polyacrylic acids such as, for example, Carbopol®, alginates, gelatine),
disintegrants (for example modified starch, carboxymethylcellulose-sodium, sodium starch glycolate such as, for example, Explotab®, cross-linked polyvinylpyrrolidon, croscarmellose-sodium such as, for example, AcDiSol®),
flow regulators, lubricants, glidant and mould release agents (for example magnesium stearate, stearic acid, talc, highly-disperse silicas such as, for example, Aerosil®),
coating materials (for example sugar, shellac) and film formers for films or diffusion membranes which dissolve rapidly or in a modified manner (for example polyvinylpyrrolidones such as, for example, Kollidon®, polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®),
capsule materials (for example gelatine, hydroxypropylmethylcellulose),
synthetic polymers (for example polylactides, polyglycolides, polyacrylates, polymethacrylates such as, for example, Eudragit®, polyvinylpyrrolidones such as, for example, Kollidon®, polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

Further excipients and procedures are described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

The components A, B and C may be administered independently of one another by the oral, intravenous, topical, local installations, intraperitoneal or nasal route.

Component A is administered intravenously, intraperitoneally, preferably it is administered orally. Component B preferably is administered by the intravenous route. Component C is administered intravenously, intraperitoneally, preferably it is administered orally.

The pharmaceutical composition (formulation) varies by the route of administration.

Components of this invention can be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

Components of this invention can also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions can be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs can be formulated with sweetening agents such as, for example, glycerol, propylene glycol, sorbitol or sucrose. Such formulations may also contain a demulcent, and preservative, such as methyl and propyl parabens and flavoring and coloring agents.

Components of this invention can also be administered parenterally, that is, subcutaneously, intravenously, intraocularly, intrasynovially, intramuscularly, or interperitoneally, as injectable dosages of the compound in preferably a pharmaceutically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions of the present invention can be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation can also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Components of the invention can also be administered in the form of suppositories for rectal administration of the drug. These components can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It can be desirable or necessary to introduce a component of the present invention to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

In accordance with another aspect, the present invention concerns the use of the combination of the present invention as described herein for the treatment or prophylaxis of a disease, preferably multiple myeloma.

In accordance with another aspect, the present invention concerns the kit as described herein for the treatment or prophylaxis of a disease, preferably multiple myeloma.

In accordance with another aspect, the present invention concerns the pharmaceutical composition as described supra for the treatment or prophylaxis of a disease, preferably multiple myeloma.

In accordance with another aspect, the present invention covers the use of such combinations as described supra for the preparation of a medicament for the treatment or prophylaxis of a disease, preferably multiple myeloma.

In accordance with another aspect, the present invention covers the use of such kit as described supra for the preparation of a medicament for the treatment or prophylaxis of a disease, preferably multiple myeloma.

In accordance with another aspect, the present invention covers the use of such pharmaceutical composition as described supra for the preparation of a medicament for the treatment or prophylaxis of a disease, preferably multiple myeloma.

In accordance with another aspect, the present invention concerns methods for the treatment and/or prophylaxis of a disease, preferably multiple myeloma, using an effective amount of the combination as described supra.

In accordance with another aspect, the present invention concerns methods for the treatment and/or prophylaxis of a disease, preferably multiple myeloma, using an effective amount of the kit or pharmaceutical composition as described supra.

In accordance with another aspect, the present invention concerns a method of treating a disease in a patient, preferably multiple myeloma, comprising

a) administering component A being bortezomib or a stereoisomer, a tautomer, an N-oxide, a hydrate, a solvate, or a pharmaceutically acceptable salt thereof, and

b) administering component B being a pharmaceutically acceptable salt of the alkaline-earth radionuclide radium-223.

In accordance with another aspect, the present invention concerns a method of treating a disease in a patient, preferably multiple myeloma, comprising

a) administering component A being bortezomib or a stereoisomer, a tautomer, an N-oxide, a hydrate, a solvate, or a pharmaceutically acceptable salt thereof, and

b) administering component B being a pharmaceutically acceptable salt of the alkaline-earth radionuclide radium-223, and optionally

c) administering component C being a pharmaceutical agent as described infra.

Preferably, an aqueous solution of radium-223 chloride (223RaCl₂) for intravenous injection, sterile and free from bacterial endotoxins is used.

Preferably, the solution is isotonic, containing a sodium citrate buffered saline to physiological pH.

Methods of Administration of Radium-223

The 223Ra salt or derivative thereof will be administered to a mammal, such as a human, in need thereof by all available administration routes, such as oral, subcutaneous, intravenous, intraarterial or transcutane. Preferably the active compound is administered by injection or infusion.

Oral administration is performed by use of tablets, capsules, powders or in liquid form, such as suspension, solution, syrup or emulsion. When formed into tablets conventional expicients, lubricating agents and binding agents are used.

When administered as liquids conventional liquid carriers are used.

When administered as injection or infusion solutions the carrier is preferably isotonic saline, with or without agent(s) to stabilize the radium cation to prevent precipitation of radium salts or insoluble complexes.

Preferably, radium-223 is administered intravenously by qualified personnel as a slow bolus injection. An intravenous access line should be used for administration of radium-223. The line should be flushed with isotonic saline before and after injection of radium-223.

Dosages of Radium-223

The concentrations of the compounds in the preparation will generally be less than the individual LD50 dose, for example less than 20% of the LD50 dose, and thus vary for the different components.

The activity of 223Ra will be dependent upon the type and route of administration and the underlying condition or disease and will vary between approximately 50 kBq to approximately 10 MBq, administered in single or multiple doses for mammals, such as for example humans.

A preferred dosage regimen for radium-223 chloride injection is 50 kBq per kg body weight given at 4 week intervals, as a course consisting of 6 injections. Single radium-223 doses up to 250 kBq per kg body weight were evaluated in a phase I clinical trial. The observed adverse reactions at this dose were diarrhea and reversible myelosuppression (including one case (1/5) of grade 3 neutropenia).

As an example, the aqueous radium-223 dichloride solution may be supplied in a single-dose 10 ml vial which contains a fill volume of 6 ml. This product has a radioactivity concentration of radium-223 of 1,000 kBq/mL (0.03 mCi/mL), corresponding to 0.53 ng/mL of radium at reference date. The active moiety is the alpha particle emitting nuclide radium 223 (half-life is 11.4 days), present as a divalent cation (223Ra2+). The fraction of energy emitted from radium-223 and its daughters as alpha-particles is 95.3%, the fraction emitted as beta-particles is 3.6%, and the fraction emitted as gamma-radiation is 1.1%. The combined energy from the emitted radiation from complete decay of radium-223 and its daughter nuclides is 28.2 MeV.

Radium-223 selectively targets areas of increased bone turnover, as in bone metastases, and concentrates by forming a complex with hydroxyapatite. Alpha emission contributes about 93% of the total radiation absorbed dose. The high linear energy alpha particle radiation induces double-strand DNA breaks, resulting in a potent and localized cytotoxic effect in the target areas containing metastatic cancer cells. The short path length (less than 100 micrometers) of the alpha particles minimizes the effect on adjacent healthy tissue such as the bone marrow.

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The present invention relates to a method for using single agent and the combinations of the present invention, in the treatment or prophylaxis of a cancer, particularly multiple myeloma. Single agent and Combinations can be utilized to inhibit, block, reduce, decrease, etc., cell proliferation and/or cell division, and/or produce apoptosis, in the treatment or prophylaxis of cancer, in particular multiple myeloma. This method comprises administering to a mammal in need thereof, including a human, an amount of a combination of this invention, or a pharmaceutically acceptable salt, isomer, polymorph, metabolite, hydrate, solvate or ester thereof; etc. which is effective for the treatment or prophylaxis of cancer, in particular multiple myeloma.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Dose and Administration

Based upon standard laboratory techniques known to evaluate compounds useful for the treatment or prophylaxis of endometrial cancer (hereinafter abbreviated to “EC”), particularly 1st line, 2nd line, relapsed, refractory, type I or type II EC, or endometriosis, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of the indication. The amount of the active agent to be administered in the treatment of the condition can vary widely according to such considerations as the particular compound and dosage unit employed, the mode of administration, the period of treatment, the age and sex of the patient treated, and the nature and extent of the condition treated.

The total amount of the active agent to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1,500 mg of active agent, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Examples

The invention is demonstrated in the following examples which are not meant to limit the invention in any way:

Proliferation assays were performed with human plasma cell leukemia (JJN-3, L-363), human multiple myeloma (LP-1, MOLP-8, RPMI-8226 and OPM-2) and mouse MM 5TGM1 cells. The corresponding in vivo effects were studied in a syngeneic 5TGM1 mouse MM model. Female C57BL/KaLwRij mice (6-9 weeks old, n=15/group) were inoculated with 5TGM1 cells via tail vein and 26 days later, radium-223 (300 kBq/kg, single iv injection) and/or bortezomib (1 mg/kg ip, twice a week) or vehicle control were administered. The development of the osteolytic lesions was detected by radiography. The hind limbs were used for histological analyses and total activity measurement performed by a gamma-counter. Osteoclast number at tumor-bone interface was measured using TRAP staining. Bortezomib inhibited proliferation of all cancer cell lines tested at 25 nM (JJN3 and OPM-2 at 2.5 nM) and radium-223 at 800 Bq/ml (L-363 and MOLP-8 at 200 Bq/ml). Additive effects were observed with combination treatment. In 5TGM1 MM model, both bortezomib and radium-223 decreased osteolytic lesion area as monotherapy (p<0.05 and p=0.01, respectively). However, the combination was more effective than either one of the monotherapies (p<0.001). Bortezomib decreased the number of osteoclasts at tumor-bone interface (p<0.05) and an additive decrease was observed with combination therapy (p<0.01) resulting in almost complete eradication of osteoclasts. Incorporation of radium-223 to bone was higher with combination therapy based on total activity measurements. All treatments were well tolerated.

In conclusion, radium-223 dichloride (Xofigo®) therapy in combination with bortezomib decreased osteolytic lesion area and almost completely eradicated osteoclasts in a mouse model of myeloma bone disease. Incorporation of radium-223 to bone matrix was improved via induction of osteoblast activity by bortezomib. These data suggest that combination radium-223 and bortezomib could be a new effective therapy in MM. Radium-223 in combination with bortezomib and dexamethasone is currently investigated in a Phase Ib/II trial in patients with early relapsed MM (NCT02928029).

1. A medicament comprising radium-223 dichloride as a sole active agent, or a pharmaceutical composition comprising radium-223 dichloride for treatment or prophylaxis of multiple myeloma.

2. A combination comprising:
a) bortezomib or a pharmaceutically acceptable salt or hydrate thereof,
b) radium-223 dichloride as a sole active agent, or a pharmaceutical composition comprising radium-223 dichloride.

3. The combination of claim 2, further comprising:
c) one or more further active agents.

4. A pharmaceutical composition which comprises a combination of:
a) bortezomib or a pharmaceutically acceptable salt or hydrate thereof; and
b) radium-223 dichloride as a sole active agent, or of a pharmaceutical composition comprising radium-223 dichloride.

5. A combination of:
a) bortezomib or a pharmaceutically acceptable salt or hydrate thereof; and
b) radium-223 dichloride as a sole active agent, or of a pharmaceutical composition containing radium-223 dichloride;
or a pharmaceutical composition comprising said combination,
adapted for preparation of a medicament for treatment or prophylaxis of multiple myeloma.

6. A kit comprising:
component A: bortezomib or a pharmaceutically acceptable salt or hydrate thereof, or a stereoisomer, a tautomer, an N-oxide, a hydrate, a solvate, or a pharmaceutically acceptable salt thereof;
component B: a pharmaceutically acceptable salt of the alkaline-earth radionuclide radium-223 or a solvate or a hydrate thereof.

7. A kit comprising:
component A: bortezomib or a pharmaceutically acceptable salt or hydrate thereof, or a stereoisomer, a tautomer, an N-oxide, a hydrate, a solvate, or a pharmaceutically acceptable salt thereof;
component B: a pharmaceutically acceptable salt of the alkaline-earth radionuclide radium-223 or a solvate or a hydrate thereof;
and, optionally,
component C: one or more, optionally one, further pharmaceutical agent(s),
in which optionally either or all of said components A, B and C in any combinations are in the form of a pharmaceutical composition which is ready for use to be administered simultaneously, concurrently, separately or sequentially.
